(19) United States
(12) Patent Application Publication
(10) Pub. No.: US 2008/0082085 A1
(43) Pub. Date: Apr. 3, 2008

(54) TIME DIVISION MULTIPLEXED, BEAM COMBINING FOR LASER SIGNAL GENERATION

(76) Inventor: NICHOLAS J. KRASUTSKY, Carrollton, TX (US)

Correspondence Address: WILLIAMS, MORGAN & AMERSON, 10333 RICHMOND, SUITE 1100, HOUSTON, TX, 77042, US

(21) Appl. No.: 11/530,213

(22) Filed: Sep. 8, 2006

(51) Int. Cl. A61B 18/18

(52) U.S. Cl. 606/2

(57) ABSTRACT

A method includes time-division multiplexing a plurality of pulsed laser signals. An apparatus includes a plurality of lasers capable of emitting pulsed laser signals; a scanner optically aligned with the laser signals; and a controller. The controller is capable of pulsing the lasers in a time-division multiplexed temporal pattern and driving the scanner to combine the time-multiplexed pulsed laser signals to generate a combined laser signal.

### BACKGROUND OF THE INVENTION

1. Field of the Invention

The present invention is directed to lasers, and, more particularly, to a technique for time-division multiplexing in beam combination.

2. Description of the Related Art

Despite a great deal of investment, attempts to develop monolithic, very high power lasers have met with only very limited success. This is generally because the technology does not scale well with the output power produced. Thermal issues tend to present the greatest challenge and therefore generally are the limiting factors. For example, higher power lasers generate larger amounts of waste heat than become increasingly difficult to dispose of as the power scales upward. But other issues become problematical as well. Non-linear effects begin to predominate, and it becomes more difficult to input the prime power to the laser. Pulsed and continuous wave laser systems suffer from similar problems.

Alternative approaches attempting to overcome these problems include coherent beam combining, wavelength division multiplexing, and geometric overlap at a point. Coherent beam combining has been effective in producing a single, diffraction limited coherent beam. However, this approach places extremely stringent requirements on the laser system and fill factor is often a problem. Open results have proven the principle, but has failed to establish the utility. Wavelength division multiplexing has a long history in the telecommunications industry, which uses low power. But, it requires tunable lasers and wavelength separation requirements limit the number of lasers. Furthermore, the beam combiner is a high loss component and is delicate (i.e., fragile, or not rugged). Geometric overlap techniques have been demonstrated to produce very high powers and are used in, for example, fusion experiments. However, the resultant beams are not suitable for directing energy at range.

Thus, in general, these alternative approaches have not produced high power laser signals at range. They generally impose stringent requirement on beam properties and achieve only modestly higher powers than the low power lasers they employ. In fact, they typically do not generate powers much higher than that which can be obtained from a single optimized laser. These inadequacies are compound in applications at long range, where the combined beam should look and act like a single, diffraction limited beam to be operationally effective. Simple, effective, long range high power laser systems still have yet to be introduced to the art.

The present invention is directed to resolving, or at least reducing, one or all of the problems mentioned above.

### SUMMARY OF THE INVENTION

The invention, in its various aspects and embodiments, is an apparatus and method for time-division multiplexing a plurality of pulsed laser signals. The apparatus, more particularly, comprises a plurality of lasers capable of emitting pulsed laser signals; a scanner optically aligned with the laser signals; and a controller. The controller is capable of pulsing the lasers in a time-division multiplexed temporal pattern and driving the scanner to combine the time-multiplexed pulsed laser signals to generate a combined laser signal.

### BRIEF DESCRIPTION OF THE DRAWINGS

The invention may be understood by reference to the following description taken in conjunction with the accompanying drawings, in which like reference numerals identify like elements, and in which:

FIG. 1 depicts selected portions of an apparatus constructed in accordance with one particular embodiment of the present invention;

FIG. 2-FIG. 3 illustrate the scanner and controller of FIG. 1 and in greater detail;

FIG. 4 illustrates the timing of the laser pulses, the mirror movement, and the combined laser signal in the embodiment of FIG. 1;

FIG. 5-FIG. 6 depict a second embodiment of the present invention scaled up from the embodiment of FIG. 1 and its timing, and mirror positions;

FIG. 7-FIG. 11 illustrate a second approach alternative to that shown in FIG. 1-FIG. 6; and

FIG. 12-FIG. 14 illustrate a third approach alternative to those shown in FIG. 1-FIG. 6 and in FIG. 7-FIG. 11.

While the invention is susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The invention disclosed herein employs a time-division multiplexing technique that allows multiple pulsed laser signals to be combined into a single, diffraction limited, pulsed beam. It directs each laser along the same path during the time when it pulses. The firings are timed to produce a single train of pulses along a common axis.

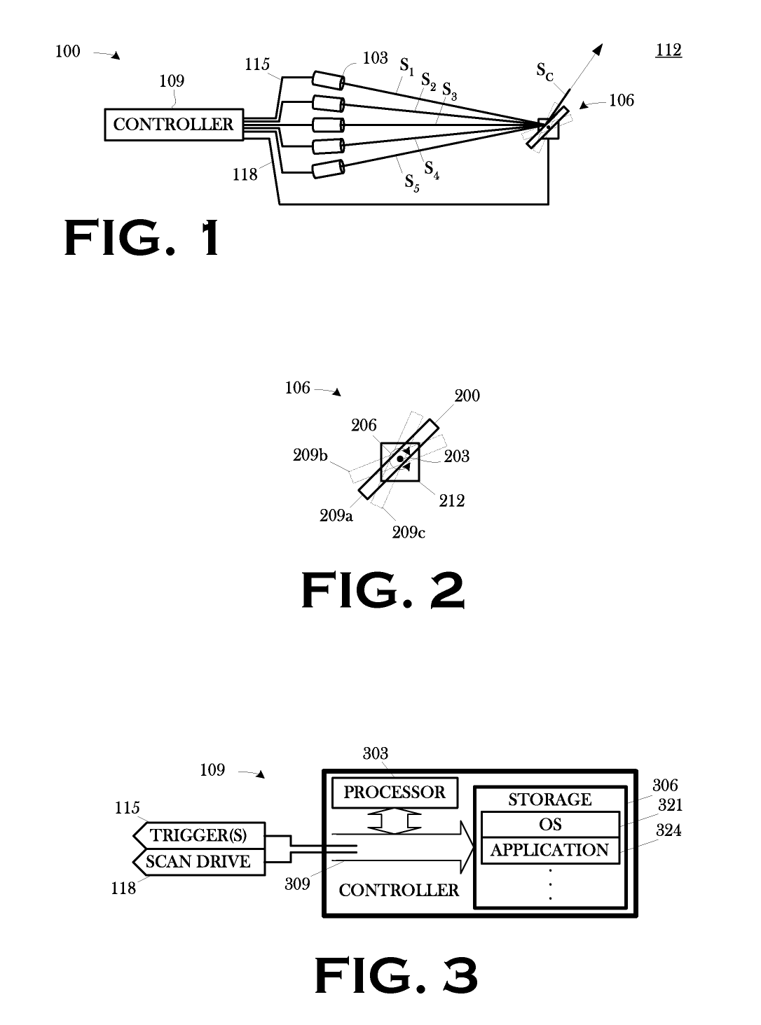

FIG. 1 illustrates one particular apparatus 100 constructed and operated in accordance with one particular embodiment of the present invention. The apparatus 100 comprises a plurality of lasers 103 (only one indicated); a scanner 106; and a controller 109. Each of the lasers 103 is capable of generating a respective pulsed laser signal S₁-S₅ when triggered by the controller 109.

Referring now to FIG. 2, the scanner 106, in this particular embodiment, scans a mirror 200 about an axis 203, as indicated by the arrow 206, that extends into and out of the page in FIG. 2. More particularly, the scanner 106 is shown in an intermediate position 209a, and is scanned through a range defined by a first position 209b and a second position 209c, both shown in broken lines.

The scanner 106 rotates through the range defined by the first and second positions 209a, 209b to align the reflections of the incident laser signals S₁-S₅ to align them on the axis of the combined laser signal S_{C}. Returning to FIG. 1, the scanner 106 reflects the incident laser signals S₁-S₅ into the field of view 112 as a combined laser signal S_{C}.

The lasers 103 may be practically any suitable pulsed laser known to the art. In the illustrated embodiment, the lasers 103 are low average power lasers. Thus, may have short, high energy, pulses but their duty cycle is low, resulting in a low average power. Or, they may have high duty cycles if their pulses are low energy pulses. Note that the number of lasers 103 is not material to the practice of the present invention although it may be an important implementation specific consideration. As will be discussed further below, the lasers 103 are all fired at the same pulse rate but their firing times are staggered so that no two pulses overlap and each pulse are separated in time by the same amount.

The scanner 106 is optically aligned with the lasers 103. Although the illustrated embodiment is optically aligned by line-of-sight, this is not necessary to the practice of the invention. Alternative embodiments may use alternative techniques well known to the art—e.g., turning mirrors, etc. In the illustrated embodiment, the mirror 200 is driven by a scanning motor 212, both shown in FIG. 2. Such scanners 106 are well known in many optical arts associated with lasers and any suitable scanner may be employed. For instance, alternative embodiments may employ one or more of an acousto-optical scanner, an electro-optical scanner, or a holographic scanner. The scanning motor 212 may be a stepper motor or a continuous motor, depending on the implementation.

Still referring to FIG. 1, the controller 109 is capable of pulsing the lasers 103 in a time-division multiplexed temporal pattern and driving the scanner 106 to combine the time-multiplexed laser pulses S₁-S₅ to generate the combined laser signal S_{C}. More particularly, the controller 109 times and transmits the triggers 115 (only one indicated) to pulse the lasers 103 in the manner described more fully below. The controller 109 also times and transmits the scan drive signal 118 to the scanner 106 to control the scanning. Pickoff detectors such as are known to the art may be employed to provide the controller with both the exact laser firing time and the mirror position.

The controller 109 may be implemented in hardware, software, or some combination of the two. FIG. 3 depicts selected portions of the controller 109, first shown in FIG. 1, in a block diagram. The controller 109 includes a processor 303 communicating with storage 305 over a bus system 309. In general, the controller 109 will handle a fair amount of data, some of which may be relatively voluminous by nature and which is processed quickly. Thus, certain types of processors may be more desirable than others for implementing the processor 303. For instance, a digital signal processor (“DSP”) may be more desirable for the illustrated embodiment than will be a general purpose microprocessor. In some embodiments, the processor 303 may be implemented as a processor set, such as a microprocessor with a mathematics co-processor.

The storage 305 may be implemented in conventional fashion and may include a variety of types of storage, such as a hard disk and/or random access memory (“RAM”). The storage 305 will typically involve both read-only and writable memory implemented in disk storage and/or cache. Parts of the storage 305 will typically be implemented in magnetic media (e.g., magnetic tape or magnetic disk) while other parts may be implemented in optical media (e.g., optical disk). The present invention admits wide latitude in implementation of the storage 305 in various embodiments. The storage 305 is also encoded with an operating system 321, and an application 324. The processor 303 runs under the control of the operating system (“OS”) 321, which may be practically any operating system known to the art.

Some portions of the detailed descriptions herein are consequently presented in terms of a software implemented process involving symbolic representations of operations on data bits within a memory in a computing system or a computing device. These descriptions and representations are the means used by those in the art to most effectively convey the substance of their work to others skilled in the art. The process and operation require physical manipulations of physical quantities. Usually, though not necessarily, these quantities take the form of electrical, magnetic, or optical signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated or otherwise as may be apparent, throughout the present disclosure, these descriptions refer to the action and processes of an electronic device, that manipulates and transforms data represented as physical (electronic, magnetic, or optical) quantities within some electronic device's storage into other data similarly represented as physical quantities within the storage, or in transmission or display devices. Exemplary of the terms denoting such a description are, without limitation, the terms “processing,” “computing,” “calculating,” “determining,” “displaying,” and the like.

Note also that the software implemented aspects of the invention are typically encoded on some form of program storage medium or implemented over some type of transmission medium. The program storage medium may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The invention is not limited by these aspects of any given implementation.

In operation, the lasers 103 run at the same frequency, but pulse at different times when triggered by the controller 109. The laser pulse lengths are short—on the order of nanoseconds through microseconds—compared to the time between pulses—which is on the order of milliseconds. Although the scanner can be stepped or continuous, the illustrated embodiment employs a stepped scanner. Each scanner position points a particular laser signal S₁-S₅ along the combined beam path while it is pulsing and then moves to the next position for the next laser signal.

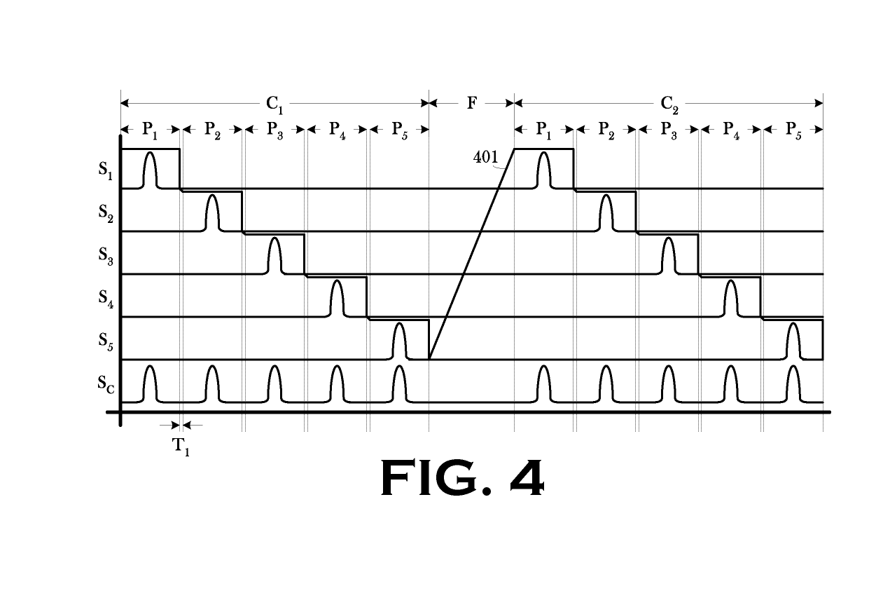

FIG. 4 illustrates the timing of the laser pulses P₁-P₅ of the laser signals S₁-S₅ produced by the lasers 103 in the embodiment of FIG. 1, the mirror position 401, and the combined laser signal S_{C}. More particularly, FIG. 4 illustrates the signals S₁-S₅ through two cycles C₁, C₂ of operation. Each laser 103 emits a respective pulse P₁-P₅ upon receiving a trigger 115 from the controller 109. Note in FIG. 4 that each of the pulses P₂-P₅ is separated by a time period T (only one, T₁, indicated) from the pulse preceding it. This time T is the time in which the scanner 106 steps from one position to the next and will be implementation specific. Note also that cycle C₂ is separated from cycle C₁ by a time period F. This time F is the flyback period in which the scanner 106 returns from the position at which pulse P₅ is combined to the position at which P₁ of the following cycle is combined.

FIG. 4 shows the pulses in the train of the combined laser signal S_{C} to be roughly contemporaneous with and of equal amplitude to the pulse P₁-P₅ of the laser signal emitted by the lasers 103. As those in the art having the benefit of this disclosure will recognize, this will not be strictly true in any given implementation. For instance, there will be some degree of offset in time spawned by the transmission time for the respective pulse P₁-P₅ to travel from the laser 103 to the scanner 106. There will also some diminishment of the energy content of the laser signal caused by, for example, interaction with the scanner—e.g., reflection loss in the illustrated embodiment. The lasers 103 may even be of different wave lengths or pulse widths provided the mirror positions are adjusted accordingly. One or more lasers can fire at a sub-multiple rate to the others, resulting in pulses which appear in the pulse train at lower rates.

However, these effects will be readily understood by those in the art given this disclosure. Furthermore, the degree of these effects will vary greatly from implementation to implementation. These layers of complexity are omitted from FIG. 4 the sake of clarity and so as not to obscure the present invention.

Thus, a plurality of laser signals (e.g., S₁-S₅) of low average power are combined to produce a combined laser signal (e.g., S_{C}) of high average power. As used herein, the term “low power” means of a power that the referenced signal may be emitted by a single laser. The term “high power” means of a power that the referenced signal is too great to be emitted from a single laser. At the current state of the art, therefore, “low power” is up to and including approximately 1 kW-2 kW, of equivalent continuous power and high power is anything over that limit. However, those in the art will appreciate that the demarcation may move as the state of the art advances.

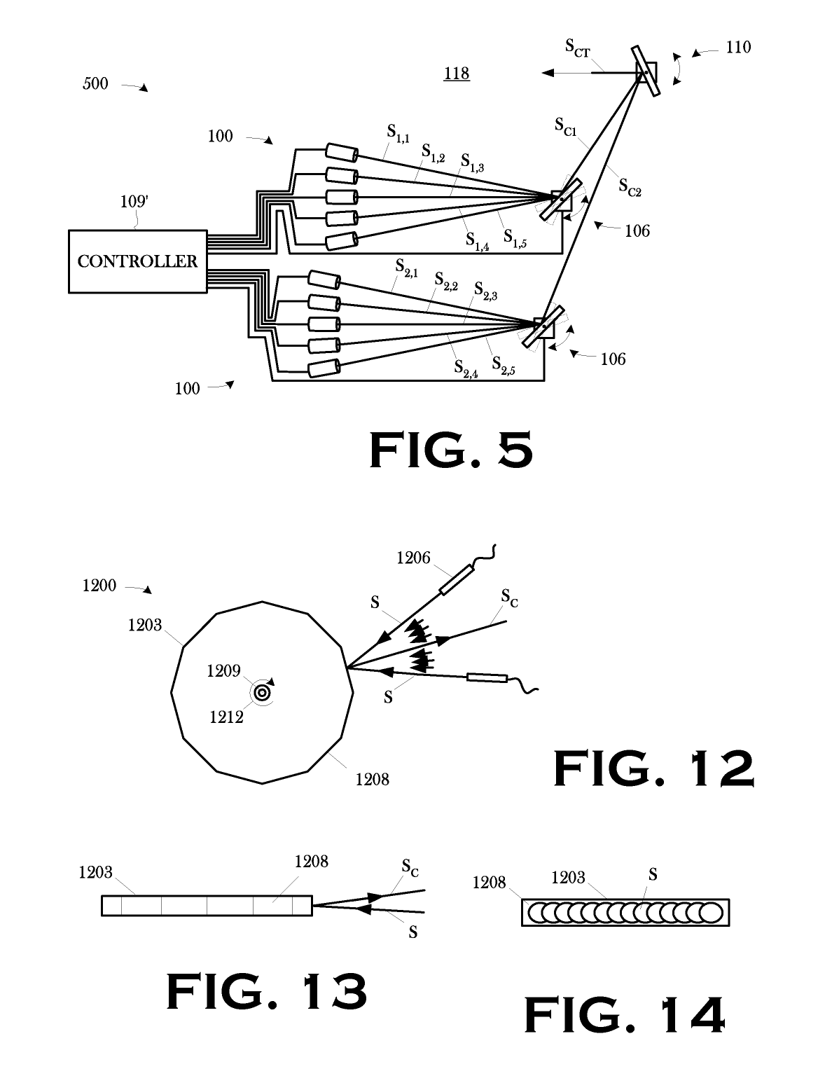

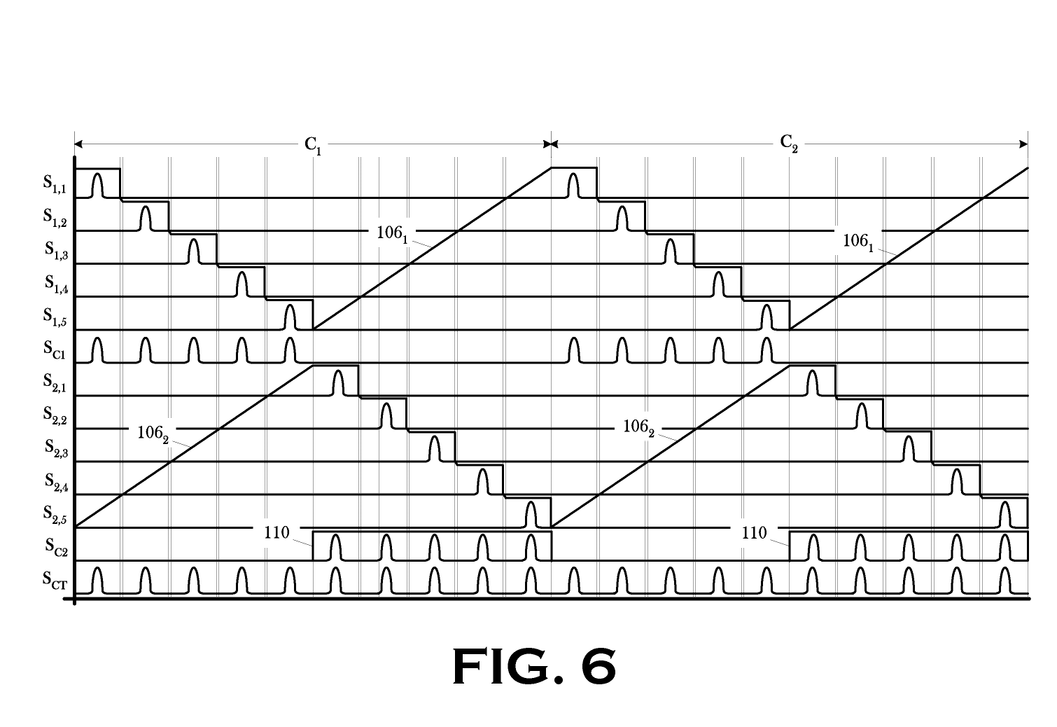

The principles of the embodiment of FIG. 1-FIG. 4 can be scaled upwardly to produce still higher power laser signals. FIG. 5-FIG. 6 illustrate a second embodiment in which the embodiment of FIG. 1 is scaled upward by employing multiple scanners 106 to combine previously combined signals. Again, two cycles C₁, C₂ are shown. Note that the flyback periods F are not shown as they are subsumed in the succeeding time periods of the other apparatus 100. Furthermore, in this example, there is no delay T between the last pulse of one combined signal and the first pulse of the second because the step delay is similarly subsumed. Using multiple scanners limits the number of steps required by an individual scanner, shortening the total travel required as well as the flyback time. In the example shown in FIG. 6, the last scanner 110 simply toggles between two positions. Using the characteristics set forth in Table 1 and Table 2 above, the combined signal S_{CT} will have a power of 13 kW.

TABLE 1
Nominal Laser Characteristics

| Characteristic | Value |
|---|---|
| Number of Beams | 10 |
| Pulse Rate | 130 Hz |
| Pulse Energy | 10 Joules |
| Pulse Length | 100 μsec |
| Pulse Jitter | <10 μsec |
| Beam Size | 5 mm |
| Wavelength | Any |
| Wavefront | Not Required |
| Matching | |

TABLE 2
Nominal Scanner Characteristics

| Characteristic | Value |
|---|---|
| Number of Steps | 10 |
| Step Size | 12 mrad |
| Step + Settle Time | 600 μsec |
| Flyback Time | 1.7 msec |
| Pointing Uncertainty | <40 μrad |

The technique can also be scaled upwardly still further, as will be discussed further below. The approach can combine a large number of laser signals, e.g., >100, but the mirror requirements go up as the number of signals and the laser pulse rate is increased. Thus, low pulse rate lasers may be preferred over high pulse rate lasers in some embodiments. However, the timing requirements for the secondary scanner will be relaxed relative to the primary mirrors. Note also that that secondary scanner must be able to handle the highest average power of the laser signals. Table 3 and Table 4 present some hypothetical characteristics for such an embodiment. Note that the combined laser signal S_{CT} in such an embodiment would have a power of 50 kW, a “high” power laser signal.

TABLE 3
Nominal Laser Characteristics

| Characteristic | Value |
|---|---|
| Number of Beams | 100 |
| Pulse Rate | 50 Hz |
| Pulse Energy | 10 Joules |
| Pulse Length | 40 μsec |
| Pulse Jitter | <10 μsec |
| Beam Size | 5 mm |
| Wavelength | Any |
| Wavefront Matching | Not Required |

TABLE 4
Nominal Scanner Characteristics

| | |
|---|---|
| Characteristic | Value |

| | | |
|---|---|---|
| Scanner Set #1 | Number of Steps | 10 |
| (10 Scanners) | Step Size | 12 mrad |
| | Step + Settle Time | 175 μsec |
| | Flyback Time | >1.5 msec |
| | Pointing Uncertainty | <40 μrad |
| Scanner #2 | Number of Steps | 10 |
| (1 Scanner) | Step Size | 12 mrad |
| | Step + Settle Time | 1.8 msec |
| | Flyback Time | 2 msec |
| | Pointing Uncertainty | <40 μrad |

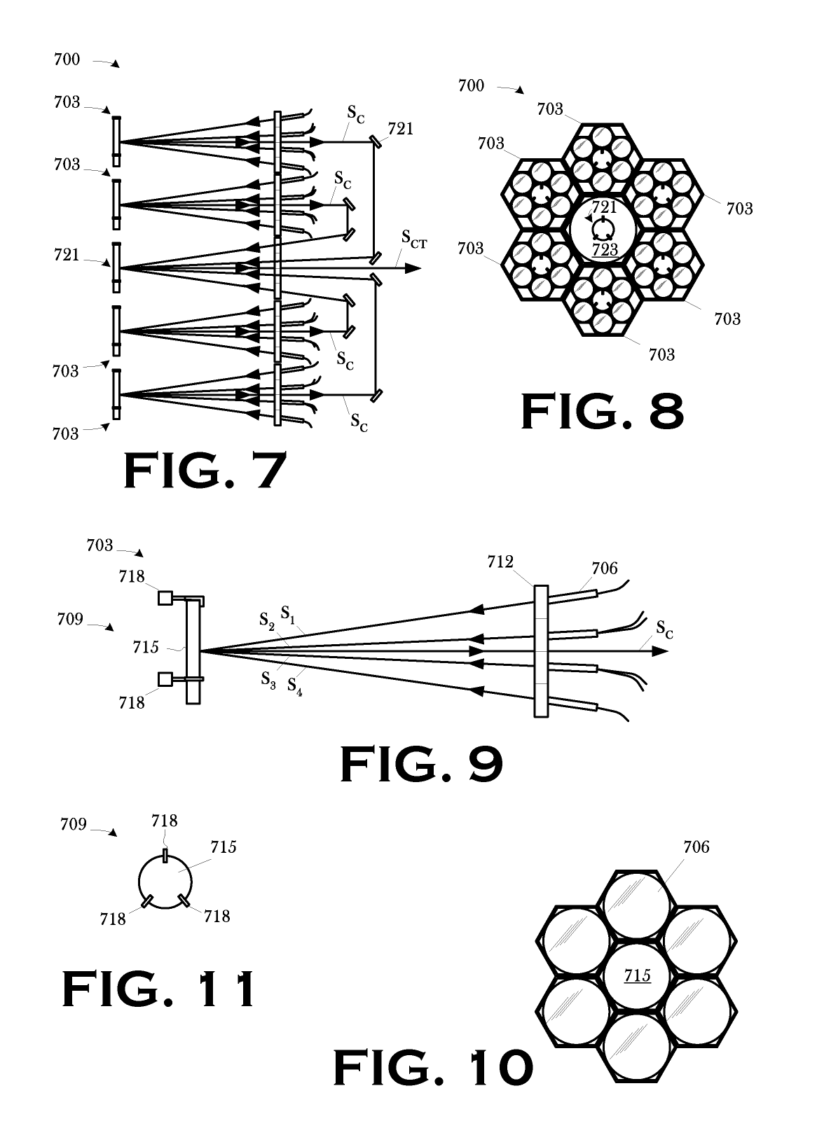

FIG. 7-FIG. 11 illustrate an alternative approach to implementing the present invention. More particularly, FIG. 7-FIG. 8 illustrate an apparatus 700 in plane side and end views, respectively. The apparatus 700 comprises a plurality of subassemblies 703—six in the illustrated embodiment. Note that not all six of the assemblies 703 are shown in FIG. 7 for the sake of clarity and so as not to obscure the present invention.

One subassembly 703 is better shown in FIG. 9-FIG. 10 in plan side and end views, respectively. In this particular embodiment, each subassembly 703 comprises a plurality of lasers 706—six in this particular embodiment—only one of which is indicated, and a scanner 709. Again, not all of the lasers 706 and the laser signals they emit are shown in FIG. 9 for the sake of clarity. The lasers 706 are mounted to and held in position by a plate 712, shown best in FIG. 9. The scanner 709 comprises a mirror 715 and a plurality of actuators 718. In operation, the lasers 706 are triggered to emit a laser signal Sₓ comprising a pulse (not shown) in a manner analogous to that discussed above relative to FIG. 4 and FIG. 6. The actuators 718 are driven to scan the mirror 715 through a range of motion to reflect the laser signals Sₓ and combine them into a combined laser signal S_{CT}.

If a larger number of actuators 718 are used around the mirror 715 the number of possible positions will double with each actuator. To use a larger number of actuators the beams would need to be closer together, or further from the mirror, or the actuator travel would need to increase. A single mirror with six actuators could be used to combine more than 60 beams

Returning to FIG. 7, each of the subassemblies 703 (only four shown) produces a combined laser signal S_{CT}. The combined laser signals S_{CT} are directed by the turning mirrors 721 (only one indicated) through an aperture 723 to another scanner 724. The scanner 724 is similar in construction and operation to the scanners 703 as described in association with FIG. 11. The scanner 703 moves through a range of motion in synchronization with the receipt of the combined laser signals S_{CT} to generate a further combined laser signal S_{CT}. This beam combining method is similar in timing and power that presented for the step scanner case except that each step is changed by moving one or more actuators.

FIG. 12-FIG. 14 illustrate a third approach to implementing the present invention. FIG. 12-FIG. 13 are plan top and side views, respectively. This particular embodiment 1200 comprises a polygonal plate 1203 and plurality of lasers 1206 (only two shown, only one indicated). The plate-shape of the polygonal plate 1203 is readily apparent in FIG. 13, but such a geometry is not necessary to the practice of the invention. The polygonal plate 1203 may be, for example, a polygonal ball in alternative embodiments. The polygonal plate 1203 includes a plurality of faces 1208, only one indicated. The dimensions of the faces 1208 are primarily a function of the size of the signals S (only two indicated) emitted from the lasers 1206 in a manner that will become apparent below.

As is best shown in FIG. 12, the polygonal plate 1203 is rotated about an axis 1209 as indicated by the arrow 1212. To sequentially present the faces 1208 for reflection of the signals S emitted by the lasers 1206. A controller (not shown) such as the controller 109 in FIG. 3 controls the rotation of the polygonal plate 1203 and the triggering of the lasers 1206, including the timing thereof. As each face 1208 is presented, each of the lasers 1206 are triggered in a sequential fashion and at staggered times such as is disclosed above relative to FIG. 4 and FIG. 6.

As is best shown in FIG. 14, each of the signals S (only one indicated) impinges upon the face 1208 at a different point and, as is shown in FIG. 12, reflected into a combined laser signal S_{CT}. Note that FIG. 14 represents a “time lapse” picture of the signals S striking the face 1208 since the pulses of the signals S are staggered to prevent them from striking all at the same time. Note also the relationship between the impingements and the dimensions of the face 1208. The face 1208 is dimensioned in both length and height so that each of the impingements can fit onto the face 1208 before the polygonal plate 1203 is rotated.

The distance from the center of rotation of a point on the face of the polygon changes with the position of the location on the face. This creates a nonlinear scan or beam wander if a single beam is scanned using a polygon scanner. This effect does not present a problem for aligning the output beam train in this invention since each laser can be positioned, aimed and timed independently. At each point in the rotation of the polygon there is an input angle and beam position on the face which will reflect along the output beam axis it is only necessary to place the lasers at these positions and time the pulses accordingly.

Consider an implementation of the embodiment in FIG. 12 whose characteristics are set forth in Table 5 below. If the beam size is (d) is 2 mm and the wavelength (λ) is 1.06 μm the beam divergence due to diffraction is 1.22λ/d or 650 μrad. If we can align the beams to within ⅕ of this diffraction limit then they will be considered a single beam. In such a system, the Maximum Image Blur (M_{irracu3}) from all sources is:

$${M}_{irracu 3} = \frac{{D}_{iv 3}}{5} = 103 µrad$$

Allowing 10 μrads for all system tolerances, the Maximum Blur (M_{blur3}) is:

M_{blur3}=M_{irracu3}−10 μrad=120 μrad

TABLE 5
Embodiment Characteristics

| Characteristic | Value |
|---|---|
| Beam Diameter | 2 mm |
| Number of Beams (N_{beam3}) | 70 |
| Wavelength (λ) | 1.06 μm |
| Average Laser Output Power (P_{ow}) | 500 W |
| Beam Diameter (B_{diam3}) | 2.0 mm |
| Pulse Length (Tₚᵤₗₛₑ) | 5.0 nsecond |
| Pulse Jitter (Tⱼᵢₜ) | 2.0 nsecond |
| Space Allowance Between Beams (Sₑₚ₃) | 0.5 mm |
| Distance from beam Row to Scanner (Rₗₐₛ₃) | 0.25 meters |
| Beam Divergence (Dᵢᵥ₃) | 650 μradian |
| Maximum Allowable Image Blur (all | 130 μradians |
| sources) (M_{irracu3}) | |
| Laser PRF (Rₑₚ) | 13.1 kHz |
| Power in Output Beam | 35 kW |

Since the blur is the angular rate times the sum of the pulse length and jitter, the maximum optical spin rate is:

$${ω}_{max 3} = \frac{{M}_{blur 3}}{{T}_{pulse} + {T}_{jit}} = 17 kradians \text{/} second$$

For 70 beams, the angular step (αₛₜₑₚ₃) for the scanner is:

$${α}_{step 3} = \frac{{B}_{diam 3} + {S}_{ep 3}}{{R}_{las 3}} = 0.01 radians$$

The total required angle to cover all beams is (θ_{scan3}) is:

θ_{scan3}=N_{beam3}αₛₜₑₚ₃=0.7 radians

Assuming a 12-sided polygon (i.e., 12 faces), the optical radians scanned per face (Φ_{face}) is:

$${Φ}_{face} = \frac{4 \prod }{12} = 1.047 radians \text{/} face$$

This gives plenty of margin to avoid the facet edges. As each face changes at the laser pulse rate, the maximum laser repetition rate (Rₘₐₓ₃) is:

$${R}_{max 3} = \frac{{ω}_{max 3}}{{Φ}_{face}} = 16200 pulses \text{/} second$$

The polygon spin rate (Pₛₚᵢₙ₃) at the maximum laser repetition rate is:

$${P}_{spin 3} = \frac{{R}_{max 3}}{12} = mrevs \text{/} sec 1350 Hz$$

### This rate is reasonable and achievable using current polygon scanner technology.

Note that these results have been verified by computer simulation. However, this embodiment of the invention is not limited to the characteristics set forth. Other embodiments may employ other characteristics.

Thus, more generally, a high speed scanner, or combination of scanners, faces a set of pulsed lasers. The lasers all fire at the same pulse rate, but their firing times are staggered. Right before each laser fires, the scanner moves into position to direct the beam along the combined beam path. When the scanner is stepped, the scanner remains in that position for the duration of the pulse, and then steps over to the next position for the firing of the next laser. This continues until all lasers have fired. The scanner then flies back to its original position and the cycle is repeated. If the scanner continuously rotates, the movement continues while each laser is firing. All the lasers fire in close sequence. The cycle is repeated at the laser pulse rate 16200 Hz, changing scanner faces as required.

The present invention therefore presents a time-division multiplexed, beam combining technique for generating a combined laser signal. Done properly, this beam combining technique allows many smaller lasers to behave like a single high power laser. The present invention therefore can be employed to produce a relatively high power laser signal from a plurality of relatively low power laser signals. Such low power lasers are a well known, proven technology. However, should one fail, the failure is not fatal to the system as a whole because other low power lasers are still functioning. The laser apparatus is therefore rugged and reliable. Furthermore, the “building block” approach disclosed above permits a high degree of scaling to meet individual, implementation specific requirements.

Note that the resultant, combined laser signal output by each of the approaches is a pulse train derived by combining a plurality pulse trains emitted at staggered times. In each of the embodiments, the at least some of the pulses are separated by some delay. In some instances, this delay results from the movement of the scanner between pulses. In some instances, this delay results from movement of the scanner between cycles (i.e., “flyback”). One advantage of this consequence is that the lasers need not all be of the same type, and some embodiments may employ multiple lasers of different types. However, the combined laser signal is diffraction limited.

It will also be appreciated by those in the art that many applications for the present invention will impose size constraints that prohibit the convenient placement of the lasers in the manner shown in the drawings hereof. As is discussed relative to the approach disclosed in FIG. 7-FIG. 11, the optical alignment of the lasers and the scanner can be achieved in ways other than by direct line-of-sight. For example, that particular embodiment employs turning mirrors to achieve the desired optical alignment. Other suitable techniques are also known to the art. Consequently, the placement of the lasers relative to the scanners is not material to the practice of the invention so long as the desired optical alignment is maintained.

This concludes the detailed description. The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. An apparatus, comprising:
a plurality of lasers capable of emitting pulsed laser signals;
a scanner optically aligned with the laser signals; and
a controller capable of pulsing the lasers in a time-division multiplexed temporal pattern and driving the scanner to pulsed laser signals.

2. The apparatus of claim 1, wherein the scanner comprises one of a scanning mirror, acousto-optical scanner, electro-optical scanner or holographic scanner.

3. The apparatus of claim 1, wherein the scanning mirror includes a mirrored, planar face.

4. The apparatus of claim 1, wherein the scanning mirror includes a mirrored, spinning, polygonal face.

5. The apparatus of claim 1, wherein the scanner is optically aligned with the lasers through direct line-of-sight positioning.

6. The apparatus of claim 1, wherein the scanner is optically aligned with the lasers through a set of turning mirrors.

7. The apparatus of claim 1 wherein the scanner comprises a mirror moved by a set of actuators.

8. The apparatus of claim 1, wherein the controller comprises a plurality of coordinated constituent controllers.

9. The apparatus of claim 1, wherein the lasers emit low average power laser signals and the time-division multiplexed laser signal is a high average power signal.

10. The apparatus of claim 1, further comprising:
a second plurality of lasers capable of emitting pulsed laser signals;
a second scanner optically aligned with the second plurality of lasers;
a third scanner optically aligned with the first and second scanners; and
wherein the controller is capable of pulsing the second plurality of lasers in a time-division multiplexed pattern with each other and with the first plurality of laser and driving the second scanner to combine the second plurality of pulsed laser signals with each other and driving the third scanner to combine the first and second time-division multiplexed laser signals.

11. The apparatus of claim 10, wherein the controller comprises a plurality of coordinated constituent controllers.

12. A method, comprising time-division multiplexing a plurality of pulsed laser signals.

13. The method of claim 12, wherein time-division multiplexing the pulsed laser signals includes:
pulsing a plurality of lasers in a time-division multiplexing temporal pattern; and
combining the pulsed laser signals.

14. The method of claim 12, wherein time-division multiplexing the pulsed laser signals further includes:
pulsing a second plurality of lasers in a time-division multiplexing temporal pattern relative to each other and to the first plurality of pulsed laser signals; and
combining the second set of laser signals; and
combining the first and second sets of time-division multiplexed pulsed laser signals.

15. The method of claim 13, wherein combining the time division-multiplexed pulsed laser signals includes actuating a scanner optically aligned with the pulsed laser signals through a range of motion to produce a combined laser signal.

16. The method of claim 12, further comprising pulsing a plurality of lasers to generate the pulsed laser signals.

17. The method of claim 12, wherein time-division multiplexing the pulsed laser signals includes scanning the time-division multiplexed pulsed laser signals into a field of view.

18. An apparatus, comprising:
means for emitting pulsed laser signals; and
means for time-division multiplexing the pulsed laser signals.

19. The apparatus of claim 18, wherein the emitting means comprises low average power lasers and the time-division multiplexed pulsed laser signal is a high average power signal.

20. The apparatus of claim 12, wherein the time-division multiplexing means includes:
means for pulsing the emitting means in a time-division multiplexing temporal pattern; and
means for combining the pulsed laser signals.

21. The apparatus of claim 20, wherein the time-division multiplexing means time-division multiplexes two sets of pulsed laser signals and the time-division multiplexes the two time-division multiplexed laser signals.

22. The apparatus of claim 20, wherein the pulsing means includes a controller capable of pulsing the emitting means in a time-division multiplexed temporal pattern.

23. The apparatus of claim 20, wherein the combining means includes:
a scanner optically aligned with the pulsed laser signals; and
a controller capable of driving the scanner to time-multiplex the pulsed laser signals.

24. The method of claim 18, wherein the time-division multiplexing means scans the time-division multiplexed pulsed laser signals into a field of view.

25. A program storage medium encoded with instructions that, when executed by a computing device, perform a method comprising time-division multiplexing a plurality of pulsed laser signals.

26. The program storage medium of claim 25, wherein time-division multiplexing the pulsed laser signals in the method includes:
pulsing a plurality of lasers in a time-division multiplexing temporal pattern; and
driving a means for combining the pulsed laser signals.

27. The program storage medium of claim 25, wherein time-division multiplexing the pulsed laser signals in the method further includes:
pulsing a second plurality of lasers in a time-division multiplexing temporal pattern relative to each other and to the first plurality of pulsed laser signals; and
combining the second set of pulsed laser signals; and
combining the first and second sets of time-division multiplexed pulsed laser signals.

28. The program storage medium of claim 25, wherein time-division multiplexing the pulsed laser signals in the method includes scanning the time-division multiplexed pulsed laser signals into a field of view.

29. A computing apparatus, comprising:
a computing device;
a bus system;
a storage communicating with the computing device over the bus system; and
a software application residing on the storage that, when executed by the computing device, time-division multiplexes a plurality of pulsed laser signals.

30. The computing apparatus of claim 29, wherein time-division multiplexing the pulsed laser signals includes:
pulsing a plurality of lasers in a time-division multiplexing temporal pattern; and
driving a means for combining the pulsed laser signals.

31. The computing apparatus of claim 29, wherein time-division multiplexing the pulsed laser signals further includes:
pulsing a second plurality of lasers in a time-division multiplexing temporal pattern relative to each other and to the first plurality of pulsed laser signals; and
combining the second set of pulsed laser signals; and
combining the first and second sets of time-division multiplexed pulsed laser signals.

32. The computing apparatus of claim 29, wherein time-division multiplexing the pulsed laser signals includes scanning the time-division multiplexed pulsed laser signals into a field of view.
